(19) United States
(12) Patent Application Publication
(10) Pub. No.: US 2020/0113778 A1
(43) Pub. Date: Apr. 16, 2020

(54) BIOPHOTONIC MASK INSTRUMENT AND CONTROLLING METHOD THEREOF

(71) Applicants: Jyh-Mei Liu Swartz, Colorado Springs, CO (US); CHIH-MIN LIU, Taoyuan City (TW)

(72) Inventors: Jyh-Mei Liu Swartz, Colorado Springs, CO (US); CHIH-MIN LIU, Taoyuan City (TW)

(21) Appl. No.: 16/653,978

(22) Filed: Oct. 15, 2019

Related U.S. Application Data
(60) Provisional application No. 62/745,488, filed on Oct. 15, 2018.

(51) Int. Cl. A61H 39/04; A61N 5/06; A61H 39/00

(52) U.S. Cl. CPC A61H 39/04; A61N 5/062; A61N 2005/0647; A61H 2205/027; A61N 2005/0651; A61H 39/002

(57) ABSTRACT

This invention is to be applied to a person's ear. The biophotonic mask instrument consists of an ear covering component, an ear attachment component, and multiple light emitting units. The biophotonic mask instrument is connected to an external intelligent electronic device with an application to control the operation of the light emitting units. The attachment component is to be clipped onto the body of the ear with the ear covering component connected to it. The multiple light emitting units are arranged to be 0.8 mm to 1.2 mm apart or 0.65 mm to 1.5 mm apart. The light emitting units are in a net design and their wave lengths are between 400 nm to 1000 nm. The biophotonic mask instrument is wirelessly connected to the intelligence device.

### FIELD OF THE DISCLOSURE

The present invention relates to a biophotonic mask instrument and its controlling method, more specifically, to a biophotonic mask instrument comprising a plurality of light emitting units and controlling method thereof.

### BACKGROUND OF THE INVENTION

Acupressure is one of the special therapeutic modalities of traditional Chinese medicine. Traditional Chinese medicine believes that there are meridians distributed all over the human body; and by being open and flowing fluently they keep the body functioning properly. They are channels for blood and energy flow. An “Acupoint” is a pivot point in meridians where the blood and energy circulate. When the pivot points are opened, the circulation of the blood and energy is unhindered. Thus, the body functions are enhanced. Massaging and stimulating the meridians and acupoints regulate meridian energy and blood flow, improve metabolism, and therefore achieve the purpose of nurturing the body and promoting health.

Auricular acupuncture is an important sub-field of Chinese acupuncture. Modern research has proven that the ear has the function of reflecting all information of the human body based on holographic mapping on the ear. The auricular points diagnosis and treatment of auricular points have gradually evolved into an independent new medical science.

As the existing instruments mostly use mechanical methods to massage and treat the muscular complaints based on acupoints, they are more geared toward the treatment for the shoulder area or the back area. There is no specific instrument developed to stimulate auricular points of the ears.

Currently, the stimulation of the auricular points of the ear is achieved by the finger pressure on the ear or attached solid objects (i.e., herbal seeds and metallic pallets) as a more invasive approach for the effect of massage. If the force of the finger is too strong or the hand is not clean, the ear can be injured or has the risk of infection.

In the early 1970s, German physiotherapist and acupuncturist Dr. Peter Mandel began using acupoints and meridian channels to introduce light into the human body. This is called acu-light therapy. This treatment approach is based on the work of some influential scientists.

German biophysicist Fritz Albert Popp provided Mandel with knowledge of human cell communication. Fritz Albert Popp proved that normal living cells emit a steady stream of photons (light particles) called biophotons. Russian physicist Dr. Sergi Pankratv's research at the Institute of Clinical and Experimental Medicine in Novosibirsk, Russia, demonstrated that human cells transmit incoming light to the body. He proved that acupuncture meridians and acupoints can coordinate the process of light entering and leaving the body.

According to the concept of light therapy, many studies have shown that low-energy lasers (non-invasive treatments) can treat most difficult pain syndromes such as fibromyalgia and tendon pain. However, the cost is relatively high to produce a low-energy laser treatment instrument. Moreover, because the laser light has a high degree of homology (coherence), all photons have the same phase and polarization, which can be superimposed to produce great intensity. Long-term repetitive use of laser treatment may also become invasive treatment.

Therefore, how to design a non-invasive health promoting instrument for the acupoints of the ear using safe light sources and requiring a lower manufacturing cost to achieve the effect of disease prevention and health enhancement is worth considering to those who have the common knowledge of this field of interest.

According to NASA research, the use of LED lights has a positive effect on wound healing and cell growth. They pointed out that near-infrared light from LEDs increases cellular energy to promote healing. Near-infrared light is mild and does not cause cell damage or entropy. Entropy is the tendency for any organized system to become chaotic and break down over time. According to Dr. IIya Prigogine (Nobel Prize thermodynamic physicist), the human body is an open system. The human body is capable of converging sunlight and other incoherent light sources to produce coherent light for the DNA in the cell. Therefore, the LED light source (incoherent light source) is a non-invasive and safe light source suitable for the design of a health promoting therapeutic device for the human body.

### SUMMARY OF THE INVENTION

The purpose of the present invention is to provide a biophotonic mask instrument and its controlling method.

As indicated in the foregoing section, current general approach for stimulation of the auricular points of the ear is achieved by mechanic and more invasive means. Such means are subject to injury of ear tissue or the risk of infection. The biophotonic mask instrument stimulates acupoints in a non-invasive manner in order to achieve the effect of disease prevention and health promotion.

In addition to utilizing a non-invasive approach, the biophotonic mask instrument requires a lower manufacture cost compared to potential alternatives such as the low-energy laser instrument and has much lower risks for long term and repetitive use by utilizing LED light source.

A biophotonic mask instrument is to be applied to one ear of the human body. The biophotonic mask instrument is wirelessly connected to an intelligent electronic device. The intelligent electronic device is located outside the biophotonic mask instrument. The biophotonic mask instrument comprises a plurality of light emitting units and an application. The plurality of light emitting units are spaced apart from each other by a fixed distance. These light emitting units are arranged in a grid shape. The wavelength of these light emitting units ranges from 400 nm to 1000 nm. Each of the light emitting units corresponds to at least one of the acupoints of the ear. An application is installed in an intelligent electronic device. The application includes a console which offers a plurality of buttons for making selections. Each of the buttons corresponds to at least one of the acupoints of the ear. Wherein when at least one of the buttons is selected, the light emitting unit corresponding to the acupoint which is corresponding to the selected button lights up.

The aforementioned biophotonic mask instrument further comprises an ear attachment component and an ear covering component. The ear attachment component is hung on the ear. The ear covering component is connected to the ear attachment component. The light emitting units are installed in the ear covering component.

Within the aforementioned biophotonic mask instrument, the fixed distance between the light emitting units is 0.8 mm to 1.2 mm or 0.65 mm to 1.5 mm.

Within the aforementioned biophotonic mask instrument, the light emitting units illuminate the earlobe of the ear with lights whose wavelengths range from 400 nm to 600 nm.

Within the aforementioned biophotonic mask instrument, the light emitting units illuminate the central region of the ear with lights whose wavelengths range from of 600 nm to 1000 nm.

Within the aforementioned biophotonic mask instrument, the light emitting unit is a light emitting diode.

A controlling method for the biophotonic mask instrument is provided. The controlling method for the biophotonic mask instrument comprises the following: Providing a plurality of light emitting units and making these light emitting units face the ear of the human body. The wavelengths of these light emitting units range from 400 nm to 1000 nm. Each of the light emitting units corresponds to at least one of acupoints of the ear. Providing an application installed in an intelligent electronic device. The application includes a console which offers a plurality of buttons for making selections. Each of the buttons corresponds to at least one of the acupoints. Selecting at least one of the buttons on the console is to light up at least one of the light emitting units. The light emitting unit corresponds to the acupoint which is corresponding to the selected button lights up.

The aforementioned controlling method for biophotonic mask instrument further comprises an ear attachment component and an ear covering component. The ear attachment component is hung on the ear. The ear covering component is connected to the ear attachment component. The light emitting units are placed in the ear covering component. These light emitting units are spaced apart from each other by a fixed distance.

Within the aforementioned controlling method for the biophotonic mask instrument, the fixed distance is 0.8 mm to 1.2 mm or 0.65 mm to 1.5 mm.

### BRIEF DESCRIPTION OF THE DRAWINGS

FIG. 1A illustrates a biophotonic mask instrument 10 in accordance with an embodiment of the present invention;

FIG. 1B illustrates a schematic diagram of one ear 8 of a human body;

FIG. 2A illustrates a schematic diagram of the biophotonic mask instrument 10 hung on the ear 8 of the human body;

FIG. 2B illustrates a schematic diagram of the biophotonic mask instrument 10 connected to an intelligent electronic device 9;

FIG. 3 illustrates the biophotonic mask instrument 10 with a different appearance;

FIG. 4 illustrates a schematic diagram of an acupoint 8H′ of an earlobe;

FIG. 5 illustrates the controlling method for biophotonic mask instrument.

### DESCRIPTION OF THE PREFERRED EMBODIMENTS

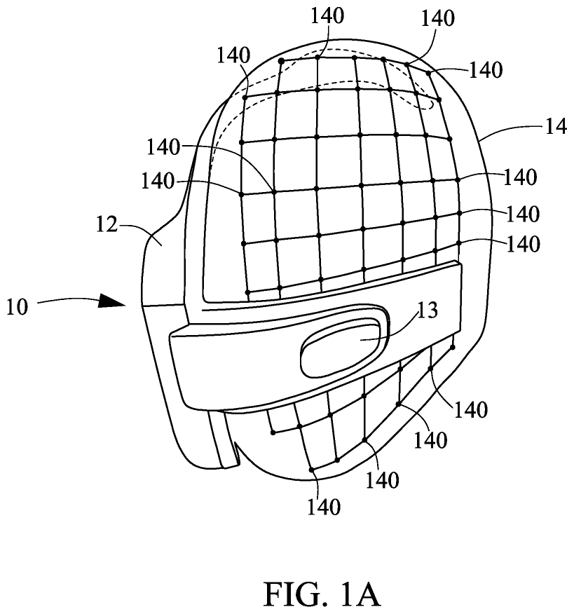

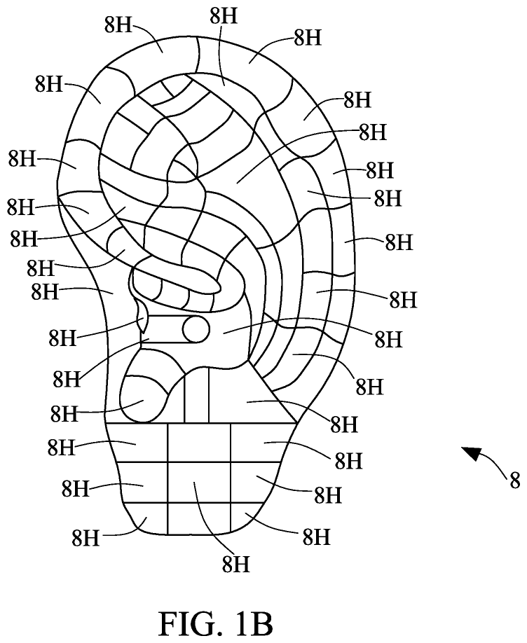

Please refer to FIG. 1A and FIG. 1B. FIG. 1A illustrates a biophotonic mask instrument 10 in accordance with an embodiment of the present invention and FIG. 1B illustrates a schematic diagram of one ear 8 of a human body. The biophotonic mask instrument 10 is applied to one ear 8 of a human body. The ear 8 includes a plurality of acupoints 8H (The twelve meridians and their branches meet and end on the ear. You can understand the conditions of the body when you stimulate the acupoints of the ear).

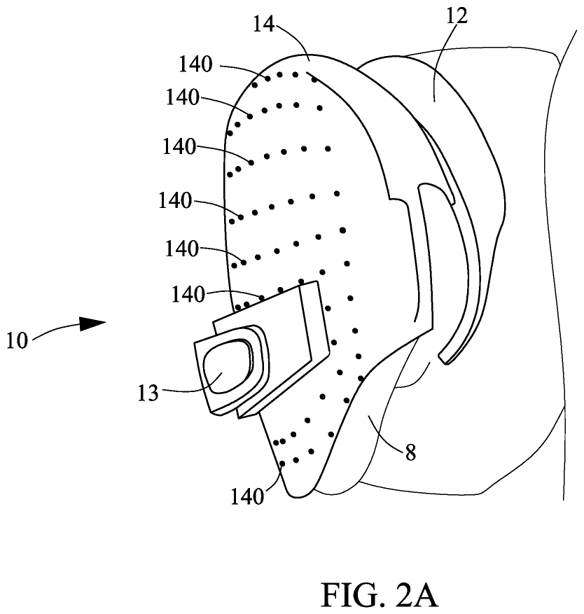

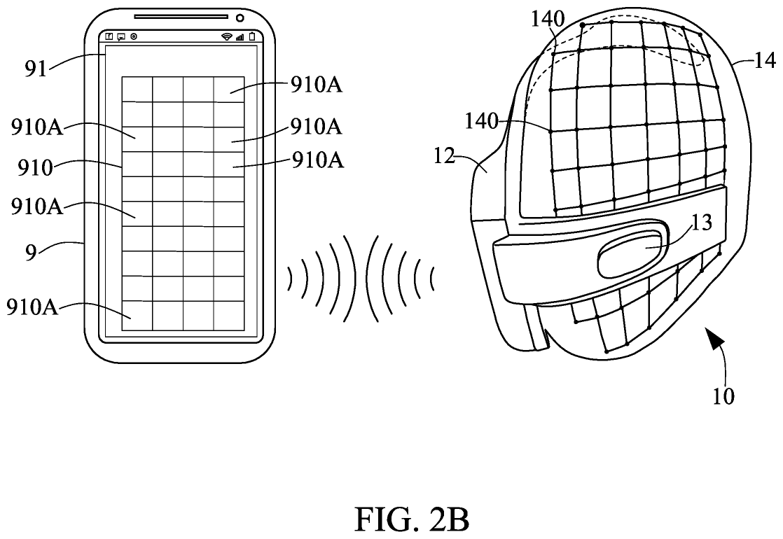

Please refer to FIG. 2A and FIG. 2B. FIG. 2A illustrates a schematic diagram of the biophotonic mask instrument 10 hung on the ear 8 of the human body. FIG. 2B illustrates a schematic diagram of the biophotonic mask instrument 10 connected to an intelligent electronic device 9. In the FIG. 2B, the biophotonic mask instrument is wirelessly connected to an intelligent electronic device 9 (e.g., smartphone). The intelligent electronic device 9 is located outside the biophotonic mask instrument. The biophotonic mask instrument comprises an ear attachment component 12, an ear covering component 14, a plurality of light emitting units 140, and an application 91. The ear covering component 14 is connected to the ear attachment component 12, and the ear covering component 14 includes a start button 13. In detail, after the start button 13 is pressed, the light emitting units 140 emit light. Please refer to FIG. 2B again, the application 91 is installed in the intelligent electronic device 9. The intelligent electronic device 9 includes a console 910. The console 910 offers a plurality of buttons 910A for making selections. Each of the buttons 910A corresponds to at least one of the acupoints 8H of the ear 8. When at least one of the buttons 910A is selected, one of the light emitting units 140 corresponding to one of the acupoints 8H corresponding to the selected button 910A lights up.

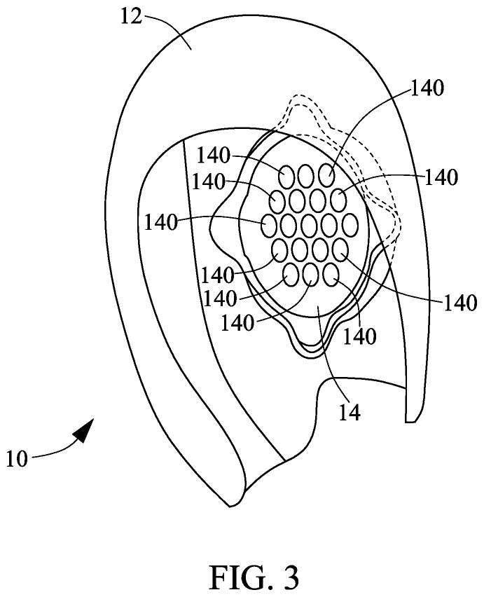

Please refer to FIG. 3. FIG. 3 illustrates the biophotonic mask instrument 10 with a different appearance. The ear covering component 14 has light emitting units 140 more concentrated in the central area of the ear 8 in the FIG. 3. These light emitting units 140 are arranged in a grid shape. The light emitting unit 140 is a light emitting diode. Therefore, in contrast to low-energy laser instrument, the manufacturing cost of the biophotonic mask instrument 10 comprising the light emitting unit 140 of the present invention is lower. In the aforementioned light emitting unites, their wavelengths range from 400 nm to 1000 nm, which provides a non-invasive way for stimulating acupoints 8H.

Furthermore, these light emitting units are spaced apart from each other by a fixed distance, and the fixed distance is from 0.8 mm to 1.2 mm or 0.65 mm to 1.5 mm. Because the ear covering component 14 abuts the ear 8, a grid-like distribution of the light-emitting units 140 covers most positions of the acupoints 8H. As the spacing between the light-emitting units 140 is small, the light emitted by the light emitting unit 140 can illuminate all the acupoints 8H.

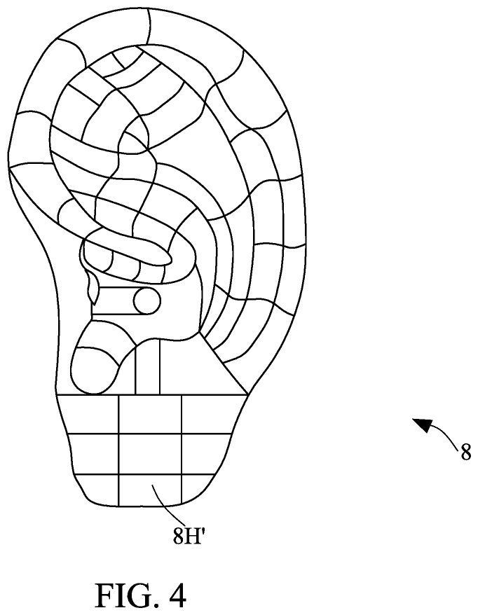

The distribution of the acupoints 8H and human organs are in a “projection” correspondence. For example, please refer to FIG. 4. FIG. 4 illustrates a schematic diagram of an acupoint 8H′ of an earlobe. The acupoint 8H′ of the earlobe corresponds to a tonsil of the human body, and inflammation of the tonsil is tonsillitis. Thus, when the light of the light emitting unit 140 radiates onto the acupoint 8H′, the inflammation of the tonsils can be prevented. As such, the light emitted by these light emitting units 140 of the biophotonic mask instrument 10 can help most of the human organs.

To elaborate, when a user of the biophotonic mask instrument 10 only wants to prevent the inflammation of the tonsil, the console 910 can control the light emitting unit 140 corresponding to the earlobe to emit light; while the light emitting units 140 corresponding to other acupoints 8H of the ear 8 are turned off. Therefore, the biophotonic mask instrument 10 can activate the corresponding light emitting units 140 in accordance with the human organs to be treated. Likewise, when a plurality of parts of the human body need to be treated, a plurality of corresponding light emitting units 140 can be simultaneously turned on. In this way, one can achieve the effects of disease prevention by stimulating acupoints using the biophotonic mask instrument.

In the example above, the light emitting unit 140 illuminates the earlobe of the ear 8 with a light of a shorter wavelength (e.g., 400 nm to 600 nm). This is because that the earlobe is located closer to the light emitting unit 140, and as such, a shorter wavelength is sufficient to achieve the desired stimulating effect. On the contrary, as the central area of the ear 8 is farther from the light emitting unit 140, the light emitting units 140 illuminate the central area of the ear 8 with a light of a longer wavelength (e.g., 600 nm to 1000 nm, infrared light) to achieve the desired stimulating effect.

In the biophotonic mask instrument, the ear covering component 14 is made of a transparent material. For example, the transparent material is a polycarbonate plastic (PC) board, an acrylic board, or a high-hardness glass. In this way, it can be clearly seen whether the light emitted by the light emitting unit 140 is accurately irradiated onto the corresponding acupoints 8H. In addition, the PC board, the acrylic board or the high-hardness glass can also ensure an overall strength of the ear covering component 14. The ear covering component 14 is not easily broken in the case of falling.

In summary, the biophotonic mask instrument of the present invention stimulates acupoints in a non-invasive manner in order to achieve the effect of disease prevention and health promotion. In addition, the manufacturing cost of the biophotonic mask instrument is lower than laser-based devices.

The controlling method for biophotonic mask instrument of the present invention could be used for auricular points of the ear or other acupoints, such as acupoints of hands.

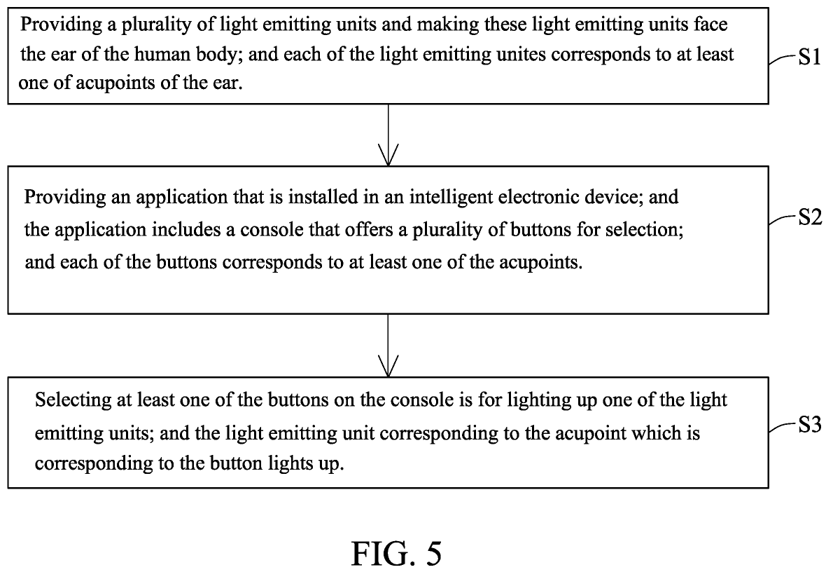

Please refer to step S1 in FIG. 5: providing a plurality of light emitting units 140 and making these light emitting units 140 face the ear 8 of the human body; each of the light emitting units 140 corresponds to at least one of acupoints 8H of the ear 8. Next, please refer to step S2: providing an application 91 that is installed in an intelligent electronic device 9; and the application includes a console 910 which offers a plurality of buttons 910A for making selections; and each of the buttons 910A corresponds to at least one of the acupoints 8H. Then, please refer to step S3: selecting at least one of the buttons 910A on the console 910 is for lighting up at least one of the light emitting units 140; and the light emitting unit corresponding to the acupoint 8H which is corresponding to the selected button 910A on the console 910 lights up.

Although the description above contains many specifics, these are merely provided to illustrate the invention and should not be construed as limitations of the scope of current invention. Thus, it will be apparent to those skilled in the art that modifications and variations can be made in the system and processes of the present invention without departing from the spirit or scope of the invention. The scope of protection of the present invention shall be determined by the scope of claims defined below.

What is claimed includes:

1. A biophotonic mask instrument applied to one ear of a human body, wherein the biophotonic mask instrument is wirelessly connected to an intelligent electronic device, and the intelligent electronic device is located outside the biophotonic mask instrument; the biophotonic mask instrument comprising:
a plurality of light emitting units spaced apart from each other by a fixed distance, and these light emitting units are arranged in a grid shape; a wavelength of a light of these light emitting units being 400 nm to 1000 nm, and each of the light emitting units corresponds to at least one of the acupoints of the ear; and
an application installed in the intelligent electronic device, wherein the application includes a console which offers a plurality of buttons for making selections, with each of the buttons corresponding to at least one of the acupoints of the ear;
wherein when at least one of the buttons is selected, the light emitting unit corresponding to the acupoint which is corresponding to the selected button lights up.

2. The biophotonic mask instrument of claim 1, further comprising:
an ear attachment component that is hung on the ear; and
an ear covering component that is connected to the ear attachment component; wherein these light emitting units are placed in the ear covering component.

3. The biophotonic mask instrument of claim 1, wherein the fixed distance is from 0.8 mm to 1.2 mm or from 0.65 mm to 1.5 mm.

4. The biophotonic mask instrument of claim 1, wherein the light emitting unit illuminates an earlobe of the ear with a light having a wavelength of 400 nm to 600 nm.

5. The biophotonic mask instrument of claim 1, wherein the light emitting unit illuminates a central region of the ear with a light having a wavelength of 600 nm to 1000 nm.

6. The biophotonic mask instrument of claim 1, wherein the light emitting unit is a light emitting diode.

7. A controlling method for biophotonic mask instrument, wherein the biophotonic mask instrument is applied to one ear of a human body; the controlling method comprising:
providing a plurality of light emitting units, and making these light emitting units face the ear of the human body; a wavelength of a light of these light emitting units being 400 nm to 1000 nm, with each of the light emitting units corresponding to at least one of acupoints of the ear;
providing an application installed in an intelligent electronic device, wherein the application includes a console which offers a plurality of buttons for making selections, and each of the buttons corresponds to at least one of the acupoints;
selecting at least one of the buttons on the console for lighting up at least one of the light emitting units, and the light emitting unit corresponding to the acupoint which is corresponding to the selected button lights up.

8. The controlling method for biophotonic mask instrument of claim 7, wherein the biophotonic mask instrument further comprising:
an ear attachment component hung on the ear; and
an ear covering component connected to the ear attachment component; wherein these light emitting units are placed in the ear covering component, and these light emitting units are spaced apart from each other by a fixed distance.

9. The controlling method for biophotonic mask instrument of claim 8, wherein the fixed distance is 0.8 mm to 1.2 mm or 0.65 mm to 1.5 mm.

10. The controlling method for biophotonic mask instrument of claim 7, wherein the light emitting unit is a light emitting diode.
